(19) United States
(12) Patent Application Publication
(10) Pub. No.: US 2025/0173871 A1
(43) Pub. Date: May 29, 2025

(54) SYSTEM AND METHOD FOR QUANTIFICATION OF DIGITIZED PATHOLOGY SLIDES

(71) Applicant: Ventana Medical Systems, Inc., Tucson, AZ (US)

(72) Inventor: Mohammad Saleh Miri, Tucson, AZ (US)

(21) Appl. No.: 19/038,628

(22) Filed: Jan. 27, 2025

Related U.S. Application Data
(63) Continuation of application No. PCT/US2023/029550, filed on Aug. 4, 2023.
(60) Provisional application No. 63/396,142, filed on Aug. 8, 2022.

(51) Int. Cl. G06T 7/00; G01N 21/78; G01N 33/574; G06V 10/764; G06V 10/766; G06V 10/77; G06V 10/82; G06V 20/69; G16H 20/10

(52) U.S. Cl. CPC G06T 7/0014; G01N 21/78; G01N 33/57492; G06V 10/764; G06V 10/766; G06V 10/7715; G06V 10/82; G06V 20/695; G06V 20/698; G16H 20/10; G01N 2333/70596; G06T 2207/20084; G06T 2207/30024; G06T 2207/30096; G06T 2207/30242; G06V 2201/03

(57) ABSTRACT

A method of determining a raw score of a pathology slide from a tissue sample includes receiving, by a regression system, a plurality of first slide features corresponding to the pathology slide, calculating, by the regression system, one or more second slide features corresponding to the pathology slide based on the plurality of first slide features, and determining, by the regression system, the raw score based on one or more features of an accumulated feature set including the plurality of first slide features and the one or more second slide features.

### CROSS-REFERENCE TO RELATED APPLICATION

This application is a bypass continuation application of International Application Number PCT/US2023/029550, filed on Aug. 4, 2023, which claims priority to and the benefit of U.S. Provisional Application 63/396,142 (“SYSTEM AND METHOD FOR QUANTIFICATION OF DIGITIZED PATHOLOGY SLIDES”), filed Aug. 8, 2022, the entire contents of all of which are incorporated herein by reference.

### FIELD

One or more aspects of some embodiments according to the present disclosure relate to quantifying a pathology slide.

### BACKGROUND

The human body's immune system utilizes T cells to help the fight infections and other diseases, including cancer. PD-L1 is a transmembrane protein that downregulates immune responses through binding to T cell's two receptors, programmed death-1 (PD-1) and B7.1. One approach to fighting cancer is blocking the PD-L1 protein, which may prevent cancer cells from inactivating T cells through both PD-1 and B7.1. A PD-L1 test helps doctors determine whether a patient is likely to benefit from cancer drugs known as immune checkpoint inhibitors. Such inhibitor drugs prevent the PD-1/PD-L1 meeting from taking place. Therefore, without receiving the “stop” signal from the PD-L1 protein, the T cells can go ahead an attack the tumor cells.

The PD-L1 (SP142) assay is an immunohistochemical (IHC) assay utilizing an anti PD-L1 rabbit monoclonal primary antibody to recognize the programmed death ligand 1 (PD-L1) protein. This assay was developed to identify patients who are most likely to respond to treatment with immune checkpoint inhibitors. However, studies have shown substantial inter-pathologist variability in the assessment of PD-L1 SP142 immunohistochemistry as a percentage, as well as the PD-L1 SP142 status (positive vs. negative). Two factors may contribute to this high inter-observer variability: 1) the assay is amplified and 2) the manual scoring guideline is cumbersome and complicated.

The above information disclosed in this Background section is only for enhancement of understanding of the background and therefore the information discussed in this Background section does not necessarily constitute prior art.

### SUMMARY

Aspects of embodiments of the present disclosure are directed to a cell-based scoring system utilizing artificial intelligence (AI) for quantifying digitized slides (e.g., PD-L1 SP142 digitized slides) from a patient sample and predicting the whole slide score percentage and thus the status of the patients (e.g., whether the patient is likely to benefit from a particular cancer drug).

According to some embodiments of the present disclosure, there is provided a method of determining a raw score of a pathology slide from a tissue sample that includes: receiving, by a regression system, a plurality of first slide features corresponding to the pathology slide; calculating, by the regression system, one or more second slide features corresponding to the pathology slide based on the plurality of first slide features; and determining, by the regression system, the raw score based on one or more features of an accumulated feature set including the plurality of first slide features and the one or more second slide features.

In some embodiments, the pathology slide is stained with PD-L1 SP142.

In some embodiments, the plurality of first slide features include at least one of: an area of a tumor region of the pathology slide; a number of stained immune cells of the pathology slide; a number of unstained immune cells of the pathology slide; a number of stained tumor cells of the pathology slide; a number of unstained tumor cells of the pathology slide; a number of other cells of the pathology slide; and a total number of cells of the pathology slide.

In some embodiments, the calculating the one or more second slide features includes: calculating at least one of a field of view (FOV) area score, a cell area score, and a cell count score.

In some embodiments, the FOV area score is expressed as:

$$FOV area score = \frac{( average size of IC + cells ) \times ( number of IC + cells )}{Area ccupied by all cells in the slide}$$

where average size of IC+ cells represents an average size of stained immune cells, and number of IC+ cells represents a number of stained immune cells of the pathology slide.

In some embodiments, the cell area score is expressed as:

$$cell area score = \frac{( average size of IC + cells ) \times ( number of IC + cells )}{Area of Tumor}$$

where average size of IC+ cells represents an average size of stained immune cells, number of IC+ cells represents a number of stained immune cells of the pathology slide, and Area of Tumor represents an area of a tumor region corresponding to the pathology slide.

In some embodiments, the cell count score is expressed as:

$$cell count score = \frac{number of IC + cells}{total number of cells}$$

where number of IC+ cells represents a number of stained immune cells of the pathology slide, and total number of cells represents a total number of cells of the pathology slide.

In some embodiments, the determining the raw score includes: providing the one or more features of the accumulated feature set to a trained regression model configured to correlate raw score values to values of the one or more features; and estimating, by the trained regression model, the raw score corresponding to the one or more features.

In some embodiments, the regression system includes a trained machine learning model configured to correlate the one or more features of the accumulated feature set to the raw score.

In some embodiments, the trained machine learning model includes one of a K-nearest neighbors (KNN) model, a support vector machine (SVM) model, a random forest (RF) model, and a multilayer perceptron (MLP) model.

In some embodiments, the method further includes: comparing the raw score with a threshold to determine efficacy of a treatment on a patient associated with the tissue sample.

In some embodiments, the method further includes: receiving, by a classifier, an image of the pathology slide; classifying, by the classifier, each cell of a plurality of cells captured in the image by identifying each cell of the plurality of cells and assigning a cell type from among a plurality of cell types to each one of the plurality of cells; and generating, by the classifier, the plurality of first slide features based on the classification of each cell.

In some embodiments, the classifier includes a convolutional neural network.

According to some embodiments of the present disclosure, there is provided a method of determining a raw score of a pathology slide from a tissue sample, the method including: receiving, by a cell-based scoring system including a processing circuit and a memory, an image of the pathology slide; classifying, by the cell-based scoring system, each cell of a plurality of cells captured in the image by providing the image to a classifier of the cell-based scoring system, the classifier being configured to identify each cell of the plurality of cells and to assign a cell type from among a plurality of cell types to each one of the plurality of cells; generating, by the cell-based scoring system, a plurality of first slide features based on the classification of each cell; and determining, by the cell-based scoring system, the raw score based on one or more features of an accumulated feature set including the plurality of first slide features.

In some embodiments, the generating the plurality of first slide features includes: counting a number of cells assigned to each cell type of the plurality of cells; and generating the plurality of first slide features based on the number of cells assigned to each cell type.

In some embodiments, the method further includes: receiving, by the cell-based scoring system, an area of a tumor region corresponding to the image, wherein the accumulated feature set further includes the area of the tumor region.

In some embodiments, the plurality of first slide features include at least one of: a number of stained immune cells of the pathology slide; a number of unstained immune cells of the pathology slide; a number of stained tumor cells of the pathology slide; a number of unstained tumor cells of the pathology slide; a number of other cells of the pathology slide; and a total number of cells of the pathology slide.

In some embodiments, the method further includes: calculating, by the cell-based scoring system, one or more second slide features corresponding to the pathology slide based on the plurality of first slide features, wherein the accumulated feature set further includes the one or more second slide features.

In some embodiments, the calculating the one or more second slide features includes: calculating at least one of a field of view (FOV) area score, a cell area score, and a cell count score.

In some embodiments, the determining the raw score includes: providing the one or more features of the accumulated feature set to a trained regression model configured to correlate raw score values to values of the one or more features; and estimating, by the trained regression model, the raw score corresponding to the one or more features.

In some embodiments, the method further includes: comparing the raw score with a threshold to determine efficacy of a treatment on a patient associated with the tissue sample.

According to some embodiments of the present disclosure, there is provided a cell-based scoring system for determining a raw score of a pathology slide from a tissue sample, the cell-based scoring system including: a classifier including a convolutional neural network configured to: receive an image of the pathology slide; classify each cell of a plurality of cells captured in the image by identifying each cell of the plurality of cells and assigning a cell type from among a plurality of cell types to each one of the plurality of cells; generate a plurality of first slide features based on the classification of each cell; a cell-based feature generator configured to calculate one or more second slide features corresponding to the pathology slide based on the plurality of first slide feature; and a regressor configured to determine the raw score based on one or more features of an accumulated feature set including the plurality of first slide features and the one or more second slide features.

### BRIEF DESCRIPTION OF THE DRAWINGS

Non-limiting and non-exhaustive embodiments according to the present disclosure are described with reference to the following figures, wherein like reference numerals refer to like parts throughout the various views unless otherwise specified.

FIG. 1 is a flow diagram illustrating various operations that may occur in a pathology context or pathology environment, according to some embodiments;

FIGS. 2A-2D illustrate the process of manually scoring the PD-L1 assay according to examples of the related art.

FIG. 3A is a block diagram illustrating the cell classifier, according to some embodiments of the present disclosure.

FIG. 3B illustrates a labeled image that identifies the different types of cells detected by the cell classifier, according to some embodiments of the present disclosure.

FIG. 4 illustrates a block diagram of a cell-based scoring system, which includes the cell classifier and a regressor, according to some embodiments of the present disclosure.

FIG. 5 is a flow diagram illustrating a process of determining a raw score of a pathology slide from a tissue sample using cell-based classification data corresponding to the pathology slide, according to some embodiments of the present disclosure.

FIG. 6 is a flow diagram illustrating a process of determining a raw score of a pathology slide from a tissue sample, according to some embodiments of the present disclosure.

### DETAILED DESCRIPTION

Hereinafter, aspects of some example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

Pathology is the medical discipline that attempts to facilitate the diagnosis and treatment of diseases by studying tissue, cell, and fluid samples of patients. In many applications, tissue samples may be collected from patients, and processed into a form that can be analyzed by physicians (e.g., pathologists), often under magnification, by physicians to diagnose and characterize relevant medical conditions based on the tissue sample.

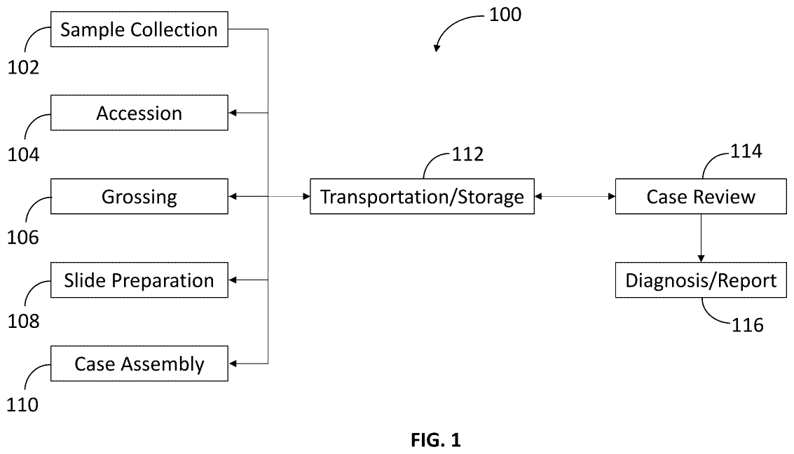

FIG. 1 is a flow diagram illustrating various operations that may occur in a pathology environment or pathology system 100. For example, when a treating physician or medical provider identifies a patient for whom an analysis of a tissue or fluid sample may be beneficial for diagnosing or treating a medical condition, a tissue or fluid sample may be collected at operation 102. The patient's identity may be collected and matched with the patient's sample, and the sample may be placed in a sterile container and/or collection medium for further processing.

The sample may then be transported to a pathology accessioning laboratory at operating 104, where the sample may be received, sorted, organized, and labeled along with other samples from other patients, for further processing.

At operation 106, the sample may be further processed as part of a grossing operation. For example, an individual tissue sample or specimen may be sliced into smaller sections for embedding and subsequent cutting for assembly onto slides.

Then, at operation 108, the sample or specimen may be mounted or deposited on one or more glass slides. The preparation of slides may involve applying one or more reagents or stains to the sample, for example, in order to improve the visibility of, or contrast between, different parts of the sample.

In some instances, at operation 110, several slides, either during the reagent or staining processing, or after the processing is completed, may be assembled or collected in a case or folio. The case may, for example, be carefully labeled with the individual patient's identifying information.

Between each of the operations 102 and 110, at operation 112, the sample, specimen, slide(s), may be transported within the medical facility, or between medical facilities (e.g., between a physician's office and a laboratory), or may be stored between processing operations.

Once the processing of the samples and slides is completed, and a pathologist is ready to review the sample, the slides and/or the case(s) holding multiple slides corresponding to the patient may again be transported, at operation 112, to the pathologist. At operation 114, the pathologist may review the slides, for example, under magnification using a microscope. An individual slide may be placed under the objective lens of the microscope, and the microscope and the slide may be manipulated and adjusted as the pathologist reviews the tissue or fluid.

Once the pathologist has completed the review of the slide, the pathologist may attempt, at operation 116, to form a medical opinion or diagnosis. Meanwhile, the sample or slides may once again be transported, at operation 112, to a longer term storage facility. In some instances, the sample or slides may be again transported, either before or after some storage period, to other physicians for further analysis, second opinions, and the like.

One example of the above-outlined operations may be performed in a pathology environment in which a pathologist identifies patients (e.g., breast cancer patients) who would likely respond to treatment with immune checkpoint inhibitors by manually analyzing and scoring the PD-L1 (SP142) assay. This immunohistochemical (IHC) assay utilizes an anti PD-L1 antibody (e.g., a rabbit monoclonal anti-PD-L1 clone SP142) to recognize the programmed death ligand 1 (PD-L1) protein in a patient's tissue sample.

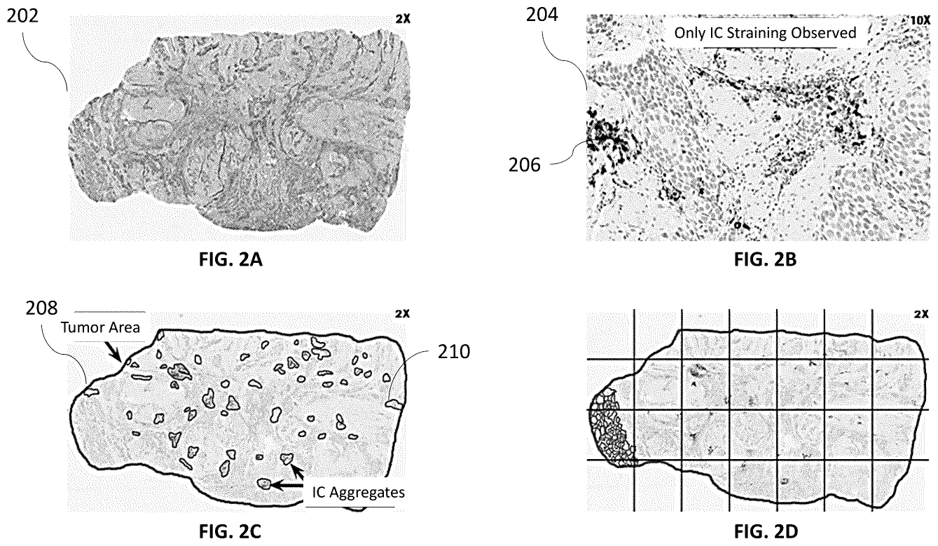

FIGS. 2A-2D illustrate the process of manually scoring the PD-L1 (e.g., PD-L1 SP142) assay according to examples of the related art.

FIG. 2A illustrates a slide 202 including a slice of a patient tissue sample containing tumor cells 202. The slide 202 may be stained with hematoxylin and eosin (H&E), which produce patterns of coloration that reveal the general layout and distribution of cells, differentiate different types of tissue, and provide a general overview of a tissue sample's structure. The pathologist may identify the viable tumor area from the H&E slide 202.

FIG. 2B illustrates an immunohistochemistry (IHC) slide 204 that includes a slice of tissue sample that is adjacent to that of the H&E slide 202 and which has PD-L1 protein (e.g., PD-L1 SP124 protein) applied to it. The pathologist may determine the presence of immunoreactivity from the IHC slide 204. The tissue slices from the IHC and H&E slides may be very close (e.g., may be about 2 μm apart) and thus may have substantially the same cell morphology. As such, the H&E slide 202 may be used by the pathologist to help identify the tumor area of the IHC slide 204 to focus on. Once the target area is identified on the IHC slide 204, the pathologist identifies any dark spots (e.g., brown spots) that may exist on the slide 204, which indicate the staining of cells (e.g., tumor or immune cells) by the PD-L1 protein. If none are found, then this is a negative sample and the patient status is identified as being negative, that is, the patient is unlikely to respond to the treatment (e.g., with immune checkpoint inhibitors). However, if dark spots (or PD-L1 stains) are observed, the pathologist then has to determine whether the dark spots are attributed to tumor cells (e.g., as a result of PD-L1 protein binding to the tumor cell membranes) or to immune cells (e.g., as a result of PD-L1 protein binding to immune cells, such as T cells). The pathologist may distinguish immune cells (ICs) from tumor cells based at least on the cell shape and size. When the concentration of immune cells that are stained with the PD-L1 protein is sufficiently high, then the patient status may be identified as being positive, that is, the patient has a high potential for responding to the treatment. That is because when the PD-L1 protein binds to immune cells (e.g., T cells) it prevents the immune cells from attacking tumor cells. A specimen having sufficiently high PD-L1-bound immune cells (i.e., assay positive immune cells) may respond well to treatment.

FIG. 2C illustrates the tumor area 208 highlighted by the pathologist and the assay positive IC regions 210 (e.g., the regions of immune cells that are stained with the PD-L1 biomarker) 210, which are also visually identified and manually highlighted by the pathologist. With these areas highlighted, the pathologist then mentally combines/aggregates the assay positive IC regions to estimate the percentage of the tumor area 208 that is occupied by the assay positive IC regions to determine the raw IC percentage score (also referred to as a “slide score”). This score may be formally expressed as

$$Slide Score = \frac{Aggregation of Assay Positive IC Regions}{Tumor Area} \qquad EQ ( 1 )$$

FIG. 2D is a visualization of the mental process that has to be performed by the pathologist to arrive at this score. Each tissue may have an associated score threshold (e.g., 1%, 5%, 10%, 20%), above which the patient status becomes positive. For instance, the cutoff for breast cancer may be 1%. Therefore, a score at or above 1% indicates that the patient is likely to respond positively to the treatment, and a score below 1% indicates that the patient is unlikely to respond to the treatment.

The complicated manual slide scoring process illustrated in FIGS. 2A-2D is cumbersome and time consuming that involves a great deal of guessing by the pathologist, which can be highly inaccurate. This results in a significant inter- and intra-observer variability in results, especially in borderline cases when the scores are near the cut-offs/thresholds. When this inaccuracy results in an incorrect determination of the patient status, and thus an incorrect treatment diagnosis, this can lead to poor or detrimental patient outcomes.

Accordingly, aspects of the present disclosure are directed to a cell-based scoring system that can reliably, repeatedly, and accurately determine the raw slide score (e.g., the PD-L1 Sp142 whole slide IC score). Further, aspects of the present disclosure are directed to a cell classifier that can identify and count the different types of cells within a tissue sample. The data generated by cell classifier may not only aid the cell-based scoring system to arrive at a slide score for a given sample, but also aid researchers in forming better hypotheses and/or testing various hypotheses about the efficacy of a particular treatment plan.

For example, by knowing the location (e.g., x-y position) and type of each cell in a tissue sample, one may calculate the average distance between the tumor cells in a sample and their closest immune cells. Such information may be relevant to why a positive patient does not respond to a particular treatment, for example. Many other relevant features may also be extracted from the raw data provided by the cell classifier which could aid searchers to better explore hypotheses about a treatment drug.

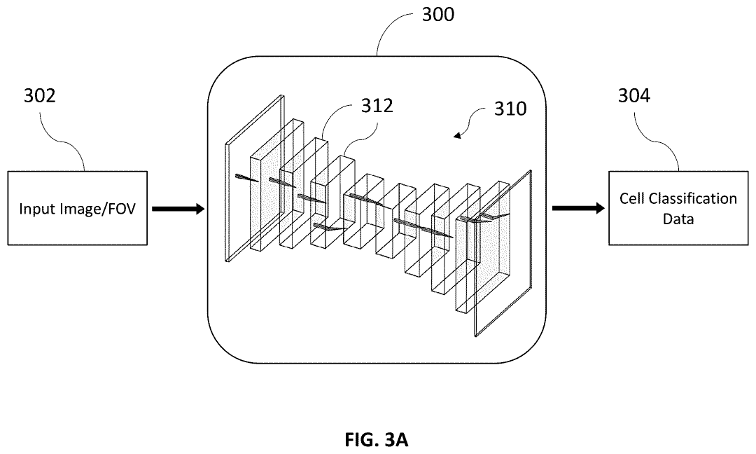

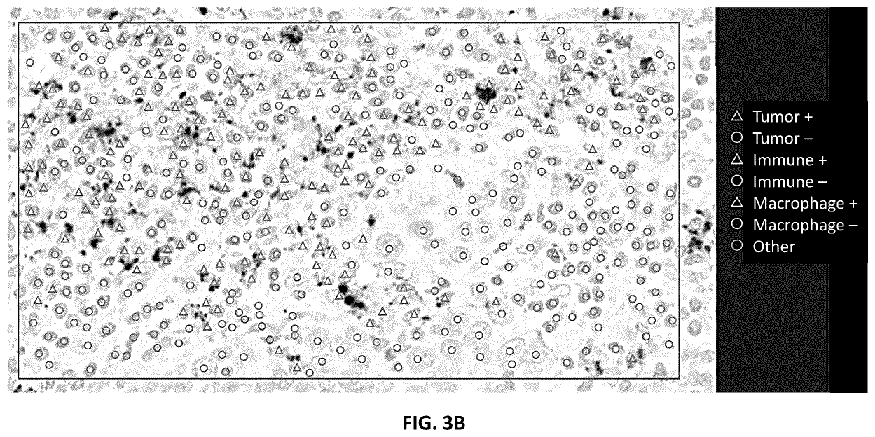

FIG. 3A is a block diagram illustrating the cell classifier 300, according to some embodiments of the present disclosure. FIG. 3B illustrates a labeled image that identifies the different types of cells detected by the cell classifier 300, according to some embodiments of the present disclosure.

Referring to FIG. 3A, in some embodiments, the cell classifier 300 receives an input image 302, which may be image of a stained tissue sample (e.g., an image of an IHC slide), detects the cells within the input image 302 (also referred to a Field of View (FOV)), and generates cell classification data 304 corresponding to the detected cells. The classification data 304 may include the type and location of each cell in the input image (e.g., a digitized red-green-blue (RGB) image) 302. The data 304 may further include the count of each identified type of cell. According to some embodiment, the types of cells classified by the cell classifier 300 may include stained immune cell (IC+), unstained immune cell (IC−), stained tumor cell (TC+), unstained tumor cell (TC−), stained macrophages (macrophage+), unstained macrophages (macrophage−), and/or other cells (that are not IC, TC, or macrophages). However, embodiments of the present disclosure are not limited thereto, and the cell classifier 300 may be trained to identify any suitable type of cell. FIG. 3B illustrates an example in which the cell classifier 300 has identified and labeled the different cells in an IHC slide.

In some embodiments, the cell classifier 300 includes a neural network (e.g., a convolutional neural network) 310 capable of cell detection and cell classification. The neural network 310 may include a number of layers each of which performs a convolutional operation, via the application of the kernels/filters, on an input feature map (IFM) 312 to generate an output feature map, which serves as the input feature map 312 of a subsequent layer. In the first layer of the neural network 310, the input feature map may be the input image 302.

The neural network 310 referred to in this disclosure may, according to some examples, be a convolutional neural network (ConvNet/CNN), which can take in an input image, assign importance (e.g., via learnable weights and biases) to various aspects/objects in the image and be able to differentiate one from the other. However, embodiments of the present disclosure are not limited thereto. For example, the neural network 310 may be a recurrent neural network (RNN) with convolution operation, or the like.

The deep learning model of the neural network 310 may be trained by providing many examples (e.g., over a hundred thousand samples) of FOVs 302 and the corresponding annotated data, which include the position of each cell on the FOV (e.g., the x-y position on the FOV of each cell) and the type (e.g., label) of each cell to the neural network 310. The annotated cell type (e.g., cell label) may be one of “IC+”, “IC−”, “TC+”, “TC−”, “macrophage+”, “macrophage−”, and “other cell”. A visualization of this annotated cell data is shown in FIG. 3B, where each cell is marked with a colored shape corresponding to the cell type.

Some or all of the cell classification data 304 may be used by a regressor to determine a raw score (e.g., the PD-L1 Sp142 whole slide IC score) for a given input image/FOV 302, and/or may be used by researchers in exploring various hypotheses about the efficacy of a particular treatment plan.

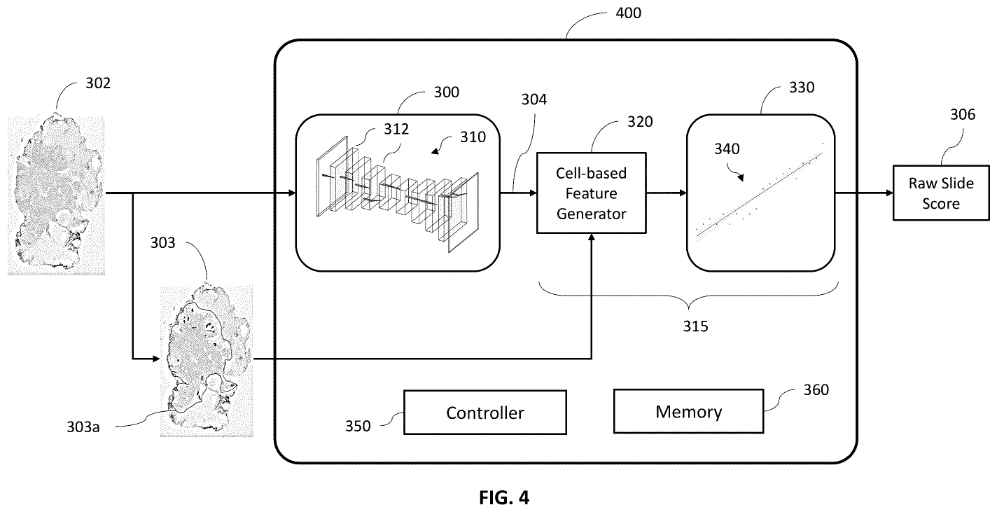

FIG. 4 illustrates a block diagram of a cell-based scoring system 400, which includes the cell classifier 300 and a regressor 330, according to some embodiments of the present disclosure.

According to some embodiments, the cell-based scoring system 400 is a sample/slide scoring system that is configured to determine the raw score of a pathology slide from a tissue sample based on an image (e.g., digitized image) of the slide. The cell classifier 300 receives an image 302 of the pathology slide and classifies each of the cells captured in the image. As described above with respect to FIG. 3, the cell classifier 300 may do so by identifying each cell in the image 302 and assigning a suitable cell type to the cell.

In some embodiments, the cell-based scoring system 400 includes a feature generator (e.g., a cell-based feature generator) 320 that is configured to generate a plurality of first slide features (e.g., a plurality of extracted features) based on the classification of each cell. The feature generator 320 and the regressor 330 may together be referred to as the regression system 315. The feature generator 320 may generate the first features by counting the number of cells that are assigned to each cell type of the plurality of cells and then generating the features based on the number of cells assigned to each cell type. For example, the first slide features may include at least one of the number of IC+ cells, the number of IC-cells, the number of TC+ cells, the number of TC-cells, the number of other cells identified within the image 302, and the total number of identified cells. In some embodiments, first slide features may also include the area of the tumor region within the image 302 of the pathology slide, which the feature generator 320 may determine based on an annotated image 303 that identifies (e.g., delineates) the viable tumor area 303a. The tumor area 303a in the annotated image 303 may be produced by (e.g., drawn/highlighted by) one or more pathologists. For example, a pathologist may outline the viable tumor region 303a in the image 302 from which the area of the tumor region may be ascertained (e.g., by the feature generator 320). In some examples, the tumor region of the annotated image 303 may represent a consensus delineation (e.g., an average) of areas identified by a number of pathologists.

In some embodiments, the feature generator 320 also determines one or more second slide features (e.g., a plurality of calculated features) corresponding to the image 302 of the pathology slide based on the first slide features. The second slide features may include a field-of-view (FOV) area score, a cell area score, and a cell count score, which are calculated based on the first slide features. For example, the FOV area score may be expressed as:

$$FOV area score = \frac{( average size of IC + cells ) \times ( number of IC + cells )}{Area ccupied by all cells in the slide} \qquad EQ ( 2 )$$

where the Area occupied by all cells in the slide is calculated by summing over all types of cells the average size of each cell type multiplied by the number of the corresponding cells. In some examples, the average immune cell size may be used as an estimate of the size of cells that fall under the “other cells” category.

Further, the cell area score may be expressed as:

$$cell area score = \frac{( average size of immune cells ) \times ( number of IC + cells )}{Area of Tumor} \qquad EQ ( 3 )$$

Here, the average size of immune cells may be a value (e.g., fixed value) that is provided to or known by the regressor 330.

Furthermore, the cell count score may be expressed as:

$$cell count score = \frac{number of IC + cells}{total number of cells} \qquad EQ ( 4 )$$

The second slide features may represent rough estimates of the actual slide score (e.g., the PD-L1 Sp142 whole slide IC score), which is defined by Equation 1 above. The first and second slide features form an accumulated feature set, one or more of which are utilized by the cell-based scoring system (e.g., the regressor 330) to determine the raw slide score 306.

According to some embodiments, the regressor 330 includes a regression model 340 that bridges the gap between the cell classification data generated by the cell classifier 300 and the raw slide score 306. The regressor 330 is trained via a regression algorithm to learn the relationship (e.g., a linear or nonlinear relationship) between one or more features of the accumulated feature set and the raw slide score. Thus, once trained, the regressor 330 can predict a raw slide score of a pathology slide based on values of the one or more features, that are supplied by the feature generator 320. Herein, the term “regression” is used informally as a general name for mathematical modeling tasks whose output is a real number. The regression model may include a K-nearest neighbors (KNN) model, a support vector machine (SVM) model, a random forest (RF) model, a multilayer perceptron (MLP) model, and/or the like. For each model, deep learning may be employed to identify a corresponding set of input features that are most relevant to the algorithm's prediction of raw slide score. For example, when the regressor 330 includes a KNN machine learning model, it may utilize the tumor area, the FOV area score, the cell area score, and the cell count score from among the accumulated feature set to predict the raw slide score. Further, when the regressor 330 includes an SVM machine learning model, it may utilize, from among the accumulated feature set, the tumor area, the number of IC+, IC−, TC+, TC−, and other cells, and the total number of cells in the pathology slide to predict the raw slide score. Furthermore, when the regressor 330 includes an RF machine learning model, it may utilize, from among the accumulated feature set, the tumor area, the number of IC+ cells, and the number of IC− cells in the pathology slide to predict the raw slide score. However, embodiments of the present disclosure are not limited thereto, and the regressor 330 may utilize any suitable set of input features from among the accumulated feature set in making its estimation/forecast of the slide score.

According to some embodiments, the regressor 330 may be a specialized AI or a general AI and is trained using training data and an algorithm, such as a back-propagation algorithm. The training data may include many examples of one or more features from among the accumulated feature set and the corresponding consensus slide score from a number of pathologists.

The regressor 330 may include a set of weights for each of the parameters of a linear regression model, or the regressor 330 may include a set of weights for connections between the neurons of a trained neural network. In some embodiments, one or more features from among the accumulated feature set are supplied to the regressor 330 as values (e.g., input features) to the input layer of the regressor 330, and the values (or a set of intermediate values) are forward propagated through the regressor 330 to generate an output, where the outputs are raw slide scores.

According to some embodiments, the cell-based scoring system 400 includes a controller 350 for controlling operations of the classifier 300, the feature generator 320, and the regressor 330, and further includes a memory 360 (e.g., an on-logic-die memory) for temporarily storing the input image 302, the outputs or intermediate results of the neural network 310 (e.g., the output feature maps generated by the layers of the neural network 310) and the regression model 340. In some examples, the memory 360 may be an embedded magneto-resistive random access memory (eMRAM), a static random access memory (SRAM), and/or the like.

For evaluating the performance of the cell-based scoring system 400 (i.e., the combination of the classifier and regressor 300 and 330), a representative benchmark containing 100 cases that closely followed the prevalence of breast cancer specimens was assembled. Three pathologists independently provided their whole slide scores for all 100 cases in the benchmark set and the median of the three scores was calculated as the consensus score for each case in the benchmark set. Table 1 is a confusion matrix on the representative benchmark set that highlights the ability of the cell-based scoring system, 400 to predict the slide score and to correctly categorize the patient as negative (i.e., having a slide score less than the 1% threshold) or positive (i.e., having a slide score greater than or equal to 1% threshold).

TABLE 1

| | | Consensus Ground-truth | |
|---|---|---|---|
| | | Negative (<1%) | Positive (1%≤) |
| Predicted | Negative (<1%) | 44 (TN) | 2 (FN) |
| | Positive (1%≤) | 0 (FP) | 54 (TP) |

As shown in the example of Table 1, the number of true positive (TP) and true negative (TN) categorizations by the cell-based scoring system 400 represent 98% of the benchmark cases, only two percent of positive cases were incorrectly (e.g., falsely) labeled as negatives (i.e., false negatives (FNs)), and there were no false positives (FPs).

The performance metrics of the cell-based scoring system 400 on the representative benchmark test are illustrated in Table 2 below.

TABLE 2

| Metric | Sensitivity | Specificity | Precision | Accuracy | F1-score |
|---|---|---|---|---|---|
| Al Algorithm | 100% | 96.43% | 95.65% | 98% | 97.78% |

The metrics in table 1 are defined as follows:

$$Sensitivity = \frac{TP}{TP + FN}$$

$$Specificity = \frac{TN}{TN + FP}$$

$$Precision = \frac{TP}{TP + FP}$$

$$Accuracy = \frac{TP + TN}{TP + FP + TN + FN}$$

$$F 1 score = 2 \times \frac{Precision \times Recall}{Precision + Recall}$$

A desirable effect of employing the regressor in the cell-based scoring system 400 is that it allows for compensating the inherent bias or correcting the systematic error that may exist in the cell classifier 300. For example, the cell classifier 300 may systematically undercount the number of IC+ cells. However, since the regressor 330 is trained on data from the cell classifier 300, the errors and biases are learned by the regressor 330 and do not translate to incorrect slide scores. In other words, any errors in the output of the cell classifier 300 are compensated by, and do not necessarily propagate through, the regressor 330. Thus, the cell-based scoring system 400 is capable of producing accurate results.

According to various embodiments of the present disclosure, the cell-based scoring system 400 is implemented using one or more processing circuits or electronic circuits configured to perform various operations as described above. Types of electronic circuits may include a central processing unit (CPU), a graphics processing unit (GPU), an artificial intelligence (AI) accelerator (e.g., a vector processor, which may include vector arithmetic logic units configured efficiently perform operations common to neural networks, such dot products and softmax), a field programmable gate array (FPGA), an application specific integrated circuit (ASIC), a digital signal processor (DSP), or the like. For example, in some circumstances, aspects of embodiments of the present disclosure are implemented in program instructions that are stored in a non-volatile computer readable memory where, when executed by the electronic circuit (e.g., a CPU, a GPU, an AI accelerator, or combinations thereof), perform the operations described. The operations performed by the cell-based scoring system 400 may be performed by a single electronic circuit (e.g., a single CPU, a single GPU, or the like) or may be allocated between multiple electronic circuits (e.g., multiple GPUs or a CPU in conjunction with a GPU). The multiple electronic circuits may be local to one another (e.g., located on a same die, located within a same package, or located within a same embedded device or computer system) and/or may be remote from one other (e.g., in communication over a network such as a local personal area network such as Bluetooth®, over a local area network such as a local wired and/or wireless network, and/or over wide area network such as the internet, such a case where some operations are performed locally and other operations are performed on a server hosted by a cloud computing service). One or more electronic circuits operating to implement the cell-based scoring system 400 may be referred to herein as a computer or a computer system, which may include memory storing instructions that, when executed by the one or more electronic circuits, implement the systems and methods described herein.

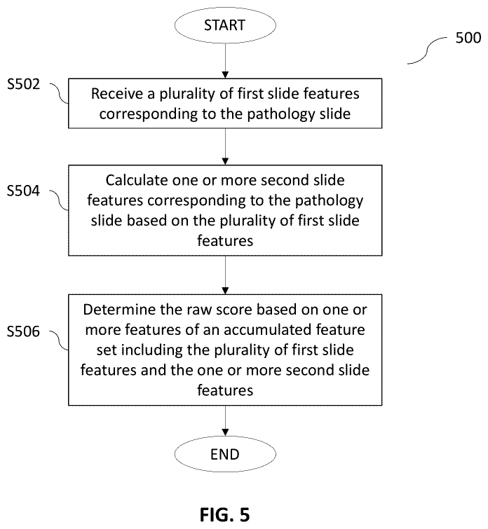

FIG. 5 is a flow diagram illustrating a process 500 of determining a raw score of a pathology slide from a tissue sample using cell-based classification data corresponding to the pathology slide, according to some embodiments of the present disclosure.

In some embodiments, the regression system 315 receives a plurality of first slide features corresponding to an image (e.g., a digitized image) 302 of a pathology slide (S502). The pathology slide may be stained with PD-L1 SP142.

The regression system 315 calculates one or more second slide features corresponding to the pathology slide based on the plurality of first slide features (S504). This may include calculating at least one of a field of view (FOV) area score, a cell area score, and a cell count score, which are defined in Equations 2-4.

The regression system 315 determines the raw score based on one or more features of an accumulated feature set including the plurality of first slide features and the one or more second slide features (S506). This may include providing the one or more features of the accumulated feature set to a trained regression model 340 configured to correlate raw score values to values of the one or more features, and estimating, by the trained regression model 340, the raw score corresponding to the one or more features.

The cell-based scoring system 400 may then compare the raw score with a threshold (e.g., 1%) to determine efficacy of a treatment on a patient associated with the tissue sample.

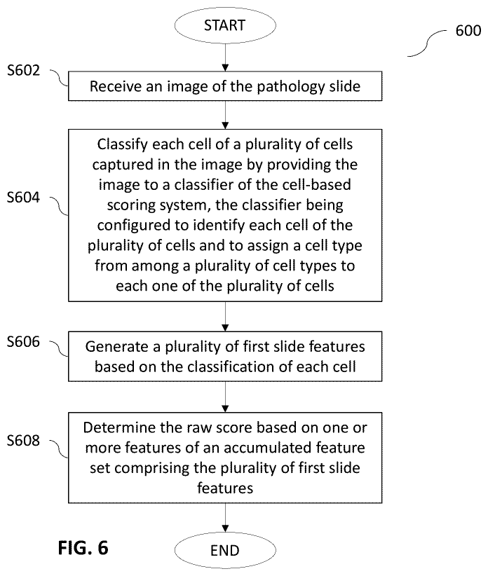

FIG. 6 is a flow diagram illustrating a process 600 of determining a raw score of a pathology slide from a tissue sample, according to some embodiments of the present disclosure.

In some embodiments, the cell-based scoring system 400 receives an image (e.g., a digitized image) 302 of the pathology slide (S602). The cell-based scoring system 400 classifies each cell of a plurality of cells captured in the image 302 by providing the image to a classifier 300, which is configured to identify each cell of the plurality of cells and to assign a cell type from among a plurality of cell types to each one of the plurality of cells (S604). The cell-based scoring system 400 then generates a plurality of first slide features based on the classification of each cell (S606), and determines the raw score based on one or more features of an accumulated feature set that includes the plurality of first slide features (S608). Generating the plurality of first slide features may include counting a number of cells assigned to each cell type of the plurality of cells, and generating the plurality of first slide features based on the number of cells assigned to each cell type. It may further include receiving an area of a tumor region 303a corresponding to the image 302 and adding (e.g., including) that in the accumulated feature set. The cell-based scoring system 400 may determine the raw score by providing the one or more features of the accumulated feature set to a trained regression model 340 that is configured to correlate raw score values to values of the one or more features; and estimating, by the trained regression model 340, the raw score corresponding to the one or more features.

The cell-based scoring system 400 may then compare the raw score with a threshold (e.g., 1%) to determine efficacy of a treatment on a patient associated with the tissue sample.

Accordingly, as described above, the cell-based scoring system has the ability to ascertain cell-level data from a digitized image of a pathology slide and to translate this cell-level data to the slide score (e.g., PD-L1 SP142 whole slide score). The cell-based scoring system is built in such a way that it not only predicts the whole slide score, but also provides invaluable cell-level information that can be used by pharmaceuticals for drug development and discoveries and exploring various hypothesis. The rapid and accurate results produced by the cell-based scoring system obviate the need for the time-consuming and cumbersome manual scoring by pathologists, which suffers from inaccuracies and significant inter- and intra-observer variability.

While some of the examples provided herein describe processing a pathology slide that is stained with PD-L1 SP142, embodiments of the present disclosure are not limited thereto, and the cell-based scoring system of the present disclosure may operate on a pathology slide that is stained with any suitable biomarker(s).

The terminology used herein is for the purpose of describing particular embodiments only and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present invention.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Although aspects of some example embodiments of the system and method of quantification of a pathology slide using a cell-based scoring system have been described and illustrated herein, various modifications and variations may be implemented, as would be understood by a person having ordinary skill in the art, without departing from the spirit and scope of embodiments according to the present disclosure. Accordingly, it is to be understood that a pathology slide manufacturing system and method according to the principles of the present disclosure may be embodiment other than as specifically described herein. The disclosure is also defined in the following claims, and equivalents thereof.

What is claimed is:

1. A method of determining a raw score of a pathology slide from a tissue sample, the method comprising:
receiving, by a regression system, a plurality of first slide features corresponding to the pathology slide;
calculating, by the regression system, one or more second slide features corresponding to the pathology slide based on the plurality of first slide features; and
determining, by the regression system, the raw score based on one or more features of an accumulated feature set comprising the plurality of first slide features and the one or more second slide features.

2. The method of claim 1, wherein the pathology slide is stained with an immunohistochemical assay.

3. The method of claim 1, wherein the plurality of first slide features comprise at least one of:
an area of a tumor region of the pathology slide;
a number of stained immune cells of the pathology slide;
a number of unstained immune cells of the pathology slide;
a number of stained tumor cells of the pathology slide;
a number of unstained tumor cells of the pathology slide;
a number of other cells of the pathology slide; and
a total number of cells of the pathology slide.

4. The method of claim 1, wherein the calculating the one or more second slide features comprises:
calculating at least one of a field of view (FOV) area score, a cell area score, and a cell count score.

5. The method of claim 4, wherein the FOV area score is expressed as:
$$FOV area score = \frac{( average size of IC + cells ) \times ( number of IC + cells )}{Area ccupied by all cells in the slide}$$
where average size of IC+ cells represents an average size of stained immune cells, and number of IC+ cells represents a number of stained immune cells of the pathology slide.

6. The method of claim 4, wherein the cell area score is expressed as:
$$cell area score = \frac{( average size of IC + cells ) \times ( number of IC + cells )}{Area of Tumor}$$
where average size of IC+ cells represents an average size of stained immune cells, number of IC+ cells represents a number of stained immune cells of the pathology slide, and Area of Tumor represents an area of a tumor region corresponding to the pathology slide.

7. The method of claim 4, wherein the cell count score is expressed as:
$$cell count score = \frac{number of IC + cells}{total number of cells}$$
where number of IC+ cells represents a number of stained immune cells of the pathology slide, and total number of cells represents a total number of cells of the pathology slide.

8. The method of claim 1, wherein the determining the raw score comprises:
providing the one or more features of the accumulated feature set to a trained regression model configured to correlate raw score values to values of the one or more features; and
estimating, by the trained regression model, the raw score corresponding to the one or more features.

9. The method of claim 1, wherein the regression system comprises a trained machine learning model configured to correlate the one or more features of the accumulated feature set to the raw score.

10. The method of claim 9, wherein the trained machine learning model comprises one of a K-nearest neighbors (KNN) model, a support vector machine (SVM) model, a random forest (RF) model, and a multilayer perceptron (MLP) model.

11. The method of claim 1, further comprising:
comparing the raw score with a threshold to determine efficacy of a treatment on a patient associated with the tissue sample.

12. The method of claim 1, further comprising:
receiving, by a classifier, an image of the pathology slide;
classifying, by the classifier, each cell of a plurality of cells captured in the image by identifying each cell of the plurality of cells and assigning a cell type from among a plurality of cell types to each one of the plurality of cells; and
generating, by the classifier, the plurality of first slide features based on the classification of each cell.

13. The method of claim 12, wherein the classifier comprises a convolutional neural network.

14. A method of determining a raw score of a pathology slide from a tissue sample, the method comprising:
receiving, by a cell-based scoring system comprising a processing circuit and a memory, an image of the pathology slide;
classifying, by the cell-based scoring system, each cell of a plurality of cells captured in the image by providing the image to a classifier of the cell-based scoring system, the classifier being configured to identify each cell of the plurality of cells and to assign a cell type from among a plurality of cell types to each one of the plurality of cells;
generating, by the cell-based scoring system, a plurality of first slide features based on the classification of each cell; and
determining, by the cell-based scoring system, the raw score based on one or more features of an accumulated feature set comprising the plurality of first slide features.

15. The method of claim 14, wherein the generating the plurality of first slide features comprises:
counting a number of cells assigned to each cell type of the plurality of cells; and
generating the plurality of first slide features based on the number of cells assigned to each cell type.

16. The method of claim 14, further comprising:
receiving, by the cell-based scoring system, an area of a tumor region corresponding to the image,
wherein the accumulated feature set further comprises the area of the tumor region.

17. The method of claim 14, wherein the plurality of first slide features comprise at least one of:
a number of stained immune cells of the pathology slide;
a number of unstained immune cells of the pathology slide;
a number of stained tumor cells of the pathology slide;
a number of unstained tumor cells of the pathology slide;
a number of other cells of the pathology slide; and
a total number of cells of the pathology slide.

18. The method of claim 14, further comprising:
calculating, by the cell-based scoring system, one or more second slide features corresponding to the pathology slide based on the plurality of first slide features,
wherein the accumulated feature set further comprises the one or more second slide features.

19. The method of claim 18, wherein the calculating the one or more second slide features comprises:
calculating at least one of a field of view (FOV) area score, a cell area score, and a cell count score.

20. The method of claim 14, wherein the determining the raw score comprises:
providing the one or more features of the accumulated feature set to a trained regression model configured to correlate raw score values to values of the one or more features; and
estimating, by the trained regression model, the raw score corresponding to the one or more features.

21. The method of claim 14, further comprising:
comparing the raw score with a threshold to determine efficacy of a treatment on a patient associated with the tissue sample.

22. A cell-based scoring system for determining a raw score of a pathology slide from a tissue sample, the cell-based scoring system comprising:
a classifier comprising a convolutional neural network configured to:
receive an image of the pathology slide;
classify each cell of a plurality of cells captured in the image by identifying each cell of the plurality of cells and assigning a cell type from among a plurality of cell types to each one of the plurality of cells;
generate a plurality of first slide features based on the classification of each cell;
a cell-based feature generator configured to calculate one or more second slide features corresponding to the pathology slide based on the plurality of first slide feature; and
a regressor configured to determine the raw score based on one or more features of an accumulated feature set comprising the plurality of first slide features and the one or more second slide features.
